(19) United States
(12) Patent Application Publication
(10) Pub. No.: US 2024/0307228 A1
(43) Pub. Date: Sep. 19, 2024

(54) METHOD FOR PROVIDING CONTROL DATA FOR AN OPHTHALMOLOGICAL LASER

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/604,899

(22) Filed: Mar. 14, 2024

(30) Foreign Application Priority Data
Mar. 15, 2023 (DE) 10 2023 106 467.3

(51) Int. Cl. A61F 9/008

(52) U.S. Cl. CPC A61F 9/00806; A61F 2009/00872

(57) ABSTRACT

A method for providing control data for an ophthalmological laser of a treatment apparatus is provided. As steps, the method comprises ascertaining a correction profile for correcting a visual disorder of a cornea from predetermined examination data; ascertaining data of a virtual postoperative cornea, which is expected by the correction by means of the correction profile, wherein the data of the virtual postoperative cornea is determined depending on a migration model, in which regrowth of an epithelial layer of the cornea is modeled; ascertaining a correction difference between an originally planned correction with the correction profile and a virtually achieved correction, which is determined from the ascertained data of the virtual postoperative cornea; adapting the correction profile depending on the migration model if the correction difference is above a preset threshold value; and providing the control data for the ophthalmological laser, which includes the adapted correction profile.

### FIELD

The present invention relates to a method for providing control data for an ophthalmological laser of a treatment apparatus. In addition, the invention relates to a treatment apparatus with at least one eye surgical laser and at least one control device for performing the method, to a computer program and to a computer-readable medium.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or photoablation in a focus situated within the organic tissue to remove a tissue from the cornea.

However, it can occur that an actually achieved correction of the cornea deviates from a planned correction and/or undesired aberrations are generated by the treatment. In particular, these effects can be ascribed to regeneration processes of the cornea.

### SUMMARY

It is the object of the present invention to improve a correction profile for the treatment of a cornea.

This object is solved by the embodiments herein. Advantageous embodiments are disclosed in the dependent claims, the following description as well as the figures.

The invention is based on the idea that regeneration processes of the cornea, in particular regrowth and shift of an epithelial layer, are modeled to thereby obtain an adapted correction profile in the treatment planning for achieving the planned correction.

An aspect of the invention relates to a method for providing control data for an ophthalmological laser of a treatment apparatus, wherein the method comprises the following steps performed by a control device. Therein, an appliance or appliance component, in particular processor or microprocessor, is meant by a control device, which is formed for executing the following method steps:

Ascertaining a correction profile for correcting a visual disorder of a cornea from predetermined examination data, ascertaining data of a virtual postoperative cornea, which is expected by the correction by means of the correction profile, are effected, wherein the data of the virtual postoperative cornea is determined depending on a migration model, in which regrowth of an epithelial layer of the cornea is modeled. Furthermore, ascertaining a correction difference between an originally planned correction by means of the correction profile and a virtually achieved correction, which is determined from the ascertained data of the virtual postoperative cornea, adapting the correction profile depending on the migration model, if the correction difference is above a preset threshold value, and providing the control data for the ophthalmological laser, which includes the adapted correction profile, are effected.

In other words, a correction profile, for example an ablation profile, can first be determined, by which a visual disorder of the cornea is to be treated. Hereto, examination data of the cornea can previously be ascertained, from which the correction profile can be planned. Subsequently, it can be simulated how a postoperative cornea looks, which has been treated with the ascertained correction profile. However, regrowth of an epithelial layer of the cornea is herein additionally simulated by a migration model, such that the virtual postoperative cornea has the state of the cornea after a regeneration process.

Herein, the migration model can have multiple assumptions about the regrowth of the epithelial layer, in particular a rate, with which the epithelial layer regrows, a rate, with which the epithelial layer is ablated, a shift of the epithelial layer in the cornea and/or a location of the regrowth of the epithelial layer. Thus, one or more of these factors can result in a function or an algorithm, which describes the migration model and which can be applied to the postoperative cornea. In particular, the regrowth of the epithelial layer can be described by means of a convolution operation, in particular a low-pass filter, which is applied to the postoperative cornea and which describes smoothing of the corneal surface towards an original shape of the cornea. In particular, a frequency response or a point spread function of the filter can be determined by the above mentioned factors.

After the virtual postoperative cornea has been modeled with a regrown epithelial layer, a correction difference between the originally planned correction, which should be generated by the initially ascertained correction profile, and the correction, which can be obtained from the ascertained data of the virtual postoperative cornea, can be determined. If such a correction difference is present, which in particular is beyond a preset threshold value, the originally planned correction profile can be adapted depending on the migration model. For example, the threshold value can be selected such that it indicates a resolution of a treatment process, for example at 0.1 micrometers. If the correction difference is below the preset threshold value, the initially planned correction profile can be used for correcting the cornea. If the correction difference is above the preset threshold value, the correction profile can be adapted such that the effect, which is described by the migration model, is compensated for. Thereto, the correction profile can for example be planned greater to compensate for the regrowth of the epithelial layer. If the migration model describes the regrowth by means of a convolution, a deconvolution operation can be applied to the data of the virtual postoperative cornea, to obtain the adapted correction profile.

Finally, control data can be provided for the ophthalmological laser, which includes the adapted correction profile. Hereto, a positioning and/or order of the laser pulses for generating the adapted correction profile can in particular be determined, for example by a further algorithm. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that the planning of correction profiles can be improved since a desired treatment result is taken into account by considering migration processes in the epithelial layer in the planning. Thus, a correction of myopia, hyperopia, astigmatism and higher order aberrations can be planned in improved manner.

The invention also includes embodiments, by which additional advantages arise.

In an embodiment a shift of the epithelial layer and an epithelial layer loss are modeled in the migration model in addition to the regrowth of the epithelial layer. This means that it is not only taken into account that the epithelial layer regrows, but also that an upper layer of the epithelium is for example ablated and the epithelial layer shifts within the cornea upon regrowth. In particular, an equilibrium of these processes can appear, which can be taken into account in the migration model. Hereby, the advantage arises that an improved simulation of the virtual postoperative cornea can be achieved.

In another embodiment, it is provided that the regrowth of the epithelial layer is modeled with a constant rate and the epithelial layer loss is modeled as proportional to a thickness of the epithelial layer. For example, the thickness of the epithelial layer can be correlated with a height of the corneal surface, wherein more loss is present with a thicker epithelial layer. In particular, an original thickness of the epithelial layer can be determined by means of ultrasonic measurements and/or OCT measurements. Hereby, the migration model can be even further improved.

In another embodiment the constant rate, by which the regrowth of the epithelial layer is modeled, is preset depending on a patient age. In particular, it can be provided that the rate decreases with increasing age. This means that the younger a patient, the faster or more the epithelial layer regrows. Hereby, the advantage arises that the migration model can be further improved.

In another embodiment the data of the virtual postoperative cornea is modeled by the migration model for a time of at least four weeks, for example three months, after treatment. In other words, a virtual postoperative cornea can be simulated by the migration model, as it looks at least four weeks after the treatment, in particular three months after the treatment. After this period of time, an equilibrium of the different effects, in particular of the regrowth and of the epithelial layer loss, can in particular be present, whereby the virtual postoperative cornea can be simpler modeled.

In another embodiment a smoothing of the cornea towards an original corneal shape is modeled by the migration model. This means, regrowth and/or shift of the epithelial layer towards a generated depression of the corneal surface occurs in the migration model, similar to a heat or mass transport or a flow of a solution along a concentration gradient, which can be described by partial differential equations. In particular, a shift of the epithelial layer from a high accumulation of epithelial cells to a recess in the cornea can occur to restore a natural shape or a smoothing. Upon a myopia correction, the epithelial layer can for example more severely regrow in the center of the cornea and outer sides upon a hyperopia correction. In particular, a smoothing constant can be assumed in the migration model, which is provided from an equilibrium between a regrowth, a shift and a loss, and thus overall indicates a way of the healing process before the epithelial layer is ablated. Therein, the smoothing constant can have a unit of length and can be regarded as the radius, over which the smoothing is effected. The smoothing constant can for example be determined by an equilibrium between the epithelial migration and epithelial loss. This means, the smoothing constant can represent a distance, over which the epithelium migrates before it peels off. Thus, a variable component of the epithelial thickness can be described, which occurs as a response to a changed surface curvature. Thus, an induced change of the depth or inclination of the cornea, in particular of the corneal surface, can be smoothed by means of the migration model. In order to achieve a change in an optical zone, a gradual transition into a transition zone, which is arranged around the optical zone, may be provided.

In another embodiment the migration model is provided by a low-pass filter, in particular a first order low-pass Butterworth filter. This means that a regrowth of the epithelial layer can be described by means of a low-pass filter as a response to a radius of curvature change by the correction profile. In particular, the virtual postoperative cornea or corneal surface can be smoothed by convolution by means of the low-pass filter to simulate the regrowth. Herein, a spatial regrowth can be represented by means of a Fourier transformation in a frequency domain for the filter. By the use of a low-pass filter, the advantage arises that high-frequency “wave-shaped” elevations can be avoided in the modeled regrowth and thus a suitable model for describing the regrowth of the epithelial layer is provided.

In another embodiment the adaptation of the correction profile is performed depending on the migration model by means of a deconvolution operation of the low-pass filter. In other words, the regrowth of the virtual postoperative cornea can first be modeled by a convolution operation with smoothing function by means of the low-pass filter, wherein the adapted correction profile can thus be ascertained by deconvolution operation of this low-pass filter to compensate for the correction difference. In particular, the deconvolution operation can be performed with a constrained iterative deconvolution algorithm. Accordingly, the effect of the regrowth, which can be modeled by means of a convolution, can be transferred to the correction profile in that the deconvolution is performed on the correction profile. Hereby, a preferred implementation for providing the adapted correction profile can be obtained.

In another embodiment the correction profile comprises an optical zone and a transition zone, wherein only the transition zone is adapted for adapting the correction profile. In other words, the transition zone can be modeled or extended or enlarged such that a regrowth only becomes noticeable in the transition zone and not in the optical zone. By the migration model, a change of the transition zone, which is situated around the optical zone, can be performed, in which a gradual change of the curvature and inclination occurs. Therein, an abrupt change of the inclination would result in an undesired peak in the radial curvature of the ascertained curvature. Accordingly, the transition zone may be designed such that a change or enlargement is not performed over a spatial frequency of 1 divided by the smoothing constant.

For example, the transition zone is adapted maximally up to a limbus, in particular up to maximally 6.5 millimeters away from a center of the optical zone. This means that a radial adaptation of the transition zone, to compensate for an effect of the regrowth, is only performed up to the limbus, thus the region between cornea and sclera, which includes the stem cells for the epithelium. In particular, the behavior of the epithelium can vary according to location in the cornea. For example, it can be assumed that the epithelium arises from limbal stem cells, whereby the peripheral epithelium is “younger”. Thus, these cells can faster peel off, faster migrate and faster divide. In particular, the peripheral epithelium can effect a more active smoothing. Thus, a width of the transition zone may be adjusted such that its component frequencies are below a radial limit frequency of 1/s rad/millimeter. Therein, hyperopic corrections can in particular have a wider transition zone since a transition contains more phases compared to a myopic correction.

For example, the adapted transition zone has a round, oval or free shape. This means that the transition zone, which can be extended for adapting the correction profile, can be formed either as a round shape or an oval shape or also as a free shape, that is for example by means of an asymmetric adaptation.

In another embodiment the adapted transition zone is extended with an additional depth of maximally 35 percent of a depth of the optical zone, in particular by 0 to 50 micrometers. For example, cubic spline curves can be used for modeling the transition zone, which offer a continuous transition both in the height and in the inclination since a discontinuity in the depth and inclination provokes healing reactions and therefore are to be avoided. Therein, a third order polynomial in radial direction is meant by a cubic spline curve, wherein a first partial derivative of the radius represents the radial slope and the second partial derivative represents an estimated value of the radial curvature. Thus, the spline coefficients can be solved for each meridian with edge conditions at the edge of the correction profile and at the edge of the optical zone by means of a system of linear algebraic equations.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume of a human or animal eye by means of optical breakthrough, in particular at least partially separate it by means of photodisruption and/or to ablate corneal layers by means of (photo)ablation and/or to effect a laser-induced refractive index change in the cornea and/or the eye lens and/or to increase a crosslinking of the cornea.

In a further advantageous configuration of the treatment apparatus according to the invention, the laser can be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 a schematic representation of a treatment apparatus according to an exemplary embodiment.

FIG. 2 a schematic method diagram for providing control data according to an exemplary embodiment.

In the figures, identical or functionally identical elements are provided with the same reference characters.

### DETAILED DESCRIPTION

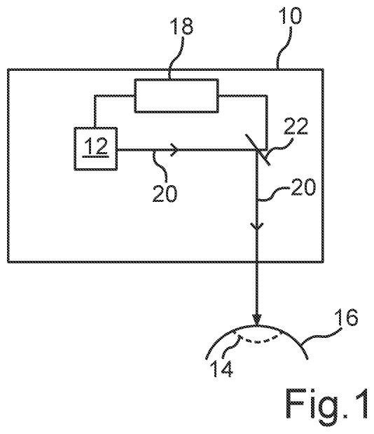

FIG. 1 shows a schematic representation of a treatment apparatus 10 with an eye surgical laser 12 for removing a tissue from a human or animal cornea 16 by means of photodisruption and/or photoablation. For example, the tissue can be provided by a correction profile 14, by which a volume body can be separated or ablated from the cornea 16 by the eye surgical laser 12 for correcting a visual disorder. This means, a geometry of the tissue to be removed can be provided by the correction profile 14, wherein the correction profile 14 can be preset to a control device 18, in particular in the form of control data, such that the laser 12 emits pulsed laser pulses in a pattern predefined by the control data into the cornea 16 of the eye to remove the tissue. Alternatively, the control device 18 can be a control device 18 external with respect to the treatment apparatus 10.

Furthermore, FIG. 1 shows that the laser beam 20 generated by the laser 12 can be deflected towards the cornea 16 by means of a beam deflection device 22, such as for example a rotation scanner, to remove the tissue. The beam deflection device 22 can also be controlled by the control device 18.

The illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

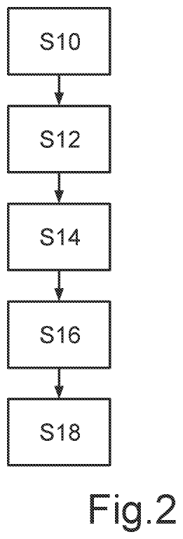

After generating the correction profile 14 in the cornea 16, it can occur that it is determined in a follow-up examination that an actually achieved correction deviates from an originally planned correction. This can be ascribed to a regeneration process of an epithelial layer of the cornea 16, which can regrow, and thus more residual tissue is present than planned by the treatment. In order to consider this effect, the method shown in FIG. 2 can be performed.

In FIG. 2, a method for providing control data for the ophthalmological laser 12 of the treatment apparatus 10 is illustrated, wherein the steps can be performed by the control device 18 of the treatment apparatus 10 or an external control device.

In a step S10, a correction profile 14 for correcting a visual disorder of the cornea 16 can be ascertained from predetermined examination data. For example, the correction profile 14 can be an ablation profile or an ablation map. Thus, the correction profile 14 provides an originally planned correction of the cornea 16. In particular, the correction profile 14 can be planned such that it coincides with an optical zone. In particular, the desired change of the cornea 16 or of the corneal surface can be indicated by the correction profile 14, wherein it can be converted into local coordinates for later deconvolution calculations. Furthermore, a depth offset can be calculated and a transition zone can be created to obtain the correction profile 14.

In a step S12, data of a virtual postoperative cornea can be determined, which is expected by the correction by means of the correction profile 14. Herein, regrowth of an epithelial layer of the cornea 16 can be simulated by means of a migration model. In particular, a regrowth of the epithelial layer, a shift of the epithelial layer within the cornea 16 and an epithelial layer loss by ablation of epithelial layer cells on the surface can be modeled in the migration model. For example, a constant rate for the regrowth of the epithelial layer can be assumed, which may be set depending on a patient age. In particular, the epithelial layer loss can be proportional to a thickness of the epithelial layer, which can for example be ascertained from the predetermined examination data, in particular from predetermined ultrasonic measurements and/or optical coherence tomography measurements. Furthermore, a state may be simulated in the migration model, in which the effects of the regrowth, the shift and the epithelial layer loss are in an equilibrium, which can for example occur four weeks after the treatment. This means that an appearance of the cornea assumed in the future can be simulated by the migration model.

For implementing the migration model, it has proven advantageous to assume it as a smoothing of the cornea towards the original corneal shape. Thereto, the virtually modeled postoperative cornea or corneal surface can be convoluted by means of a low-pass filter, in particular a first order Butterworth filter, to obtain the virtual postoperative cornea including the regrown epithelial layer. Therein, a characteristic frequency and a decline of the filter can be determined by the previously mentioned equilibrium between the constant rate, which describes the regrowth of the epithelial layer, the shift of the epithelial layer and/or the epithelial layer loss.

In a step S14, it can then be determined if a difference (correction difference) between the originally planned correction and a virtually achieved correction, which is determined from the ascertained data of the virtual postoperative cornea, is present. In other words, it can be determined if the virtual postoperative cornea looks like the originally planned cornea, wherein a deviation therefrom represents a correction difference.

If a correction difference should not be present, which usually is not the case, the originally planned correction profile 14 can be used for treating the cornea 16, wherein for the more probable case that a correction difference is present and it is in particular above a preset threshold value, which can for example be preset as a maximally tolerable deviation, the originally planned correction profile 14 can be adapted in a step S16.

Since it is known from the migration model, how the epithelial layer prospectively regrows, the adaptation can thereto be performed depending on the migration model. In particular, in using a low-pass filter as the migration model, the regrowth or the smoothing effect of the cornea 16 can be calculated by deconvolution operation of the low-pass filter with the correction profile. Thereto, a constrained iterative deconvolution algorithm may be used.

For example, a depth offset can be calculated here too and a transition zone can be modeled, wherein only the transition zone may be adapted by the deconvolution operation of the migration model in that it is adapted maximally up to a limbus and is extended in the depth by a maximum portion of 25 percent of the depth of the optical zone.

The examination of the correction difference and the adaptation of the correction profile may then be repeated until the planned cornea and the virtual postoperative cornea have the same shape and the correction difference approximates to a constant value. Therein, the maximally tolerable difference may be less than the resolution of the correction process, for example 0.1 micrometers. Thus, the last iteration results in an adapted correction profile, which comes very close to an ideal, which is required for the target correction.

Finally, control data can be provided for the ophthalmological laser 12 in a step S18, which includes the adapted correction profile. This means that the adapted correction profile can be converted into global coordinates for calculating the positioning and laser pulse sequence.

This algorithm can for example also be used for correcting higher order aberrations. Furthermore, this algorithm can also be converted such that estimations of a postoperative corneal surface, for example for photodisruptive methods, can be used instead of an ablation volume. Since the algorithm comprises a deconvolution, which can for example result in complex features like additional bends at the edge of the optical zone, an enlargement of the transition zone, a reduction of the smoothing constant s and/or pre-filtering of high-frequency components from the ablation map may be performed before application of the deconvolution.

Overall, the examples show, how a smoothing model for an epithelium of a cornea can be taken into account in the treatment planning.

1. A method for providing control data for an ophthalmological laser of a treatment apparatus, wherein the method comprises the following steps performed by a control device:
ascertaining a correction profile for correcting a visual disorder of a cornea from predetermined examination data;
ascertaining data of a virtual postoperative cornea, which is expected by a correction with the correction profile, wherein the data of the virtual postoperative cornea is determined depending on a migration model, in which regrowth of an epithelial layer of the cornea is modeled;
ascertaining a correction difference between an originally planned correction with the correction profile and a virtually achieved correction, which is determined from the ascertained data of the virtual postoperative cornea;
if the correction difference is above a preset threshold value, adapting the correction profile depending on the migration model;
providing the control data for the ophthalmological laser, which includes the adapted correction profile.

2. The method according to claim 1, wherein a shift of the epithelial layer and an epithelial layer loss are modeled in the migration model in addition to the regrowth of the epithelial layer.

3. The method according to claim 2, wherein the regrowth of the epithelial layer is modeled with a constant rate and the epithelial layer loss is modeled as proportional to a thickness of the epithelial layer.

4. The method according to claim 3, wherein the constant rate, by which the regrowth of the epithelial layer is modeled, is preset depending on a patient age.

5. The method according to claim 1, wherein the data of the virtual postoperative cornea is modeled by the migration model for a time of at least 4 weeks after treatment.

6. The method according to claim 1, wherein a smoothing of the cornea towards an original corneal shape is modeled by the migration model.

7. The method according to claim 1, wherein the migration model is provided by a low-pass filter, in particular a first order low-pass Butterworth filter.

8. The method according to claim 7, wherein the adaptation of the correction profile is performed depending on the migration model by a deconvolution operation of the low-pass filter.

9. The method according to claim 1, wherein the correction profile comprises an optical zone and a transition zone, wherein only the transition zone is adapted for adapting the correction profile.

10. The method according to claim 9, wherein the transition zone is adapted maximally up to a limbus, in particular up to maximally 6.5 mm away from a center of the optical zone.

11. The method according to claim 9, wherein the adapted transition zone has a round, oval or free shape.

12. The method according to claim 9, wherein the adapted transition zone is extended with an additional depth of maximally 35% of a depth of the optical zone, in particular by 0 to 50 μm.

13. A method for controlling a treatment apparatus, wherein the method comprises the steps of the method according to claim 1, and
transferring the provided control data to a respective ophthalmological laser of the treatment apparatus.

14. A control device configured to perform the method according to claim 1.

15. A treatment apparatus with at least one ophthalmological laser for treating a cornea of a human or animal eye by means of optical breakthrough, in particular by means of photodisruption and/or photoablation, the treatment apparatus comprising at least one control device according to claim 14.

16. A non-transitory computer-readable medium configured for storing a computer program, the computer program comprising commands which cause a treatment apparatus to execute a method according to claim 1.

17. (canceled)
